(19) United States
(12) Patent Application Publication
(10) Pub. No.: US 2021/0393767 A1
(43) Pub. Date: Dec. 23, 2021

(54) METHOD OF ENHANCING IMMUNE RESPONSE IN HOSTS

(71) Applicant: Reber Genetics Co., Ltd., Taipei City (TW)

(72) Inventors: Chia-Jung Chang, Kaohsiung City (TW); Jen-Yu Chang, Taoyuan City (TW); Cheng-Xin Yang, Taoyuan City (TW)

(73) Assignee: Reber Genetics Co., Ltd., Taipei City (TW)

(21) Appl. No.: 17/465,855

(22) Filed: Sep. 3, 2021

Related U.S. Application Data
(62) Division of application No. 16/801,175, filed on Feb. 26, 2020.
(60) Provisional application No. 62/825,834, filed on Mar. 29, 2019.

(51) Int. Cl. A61K 39/12; C12N 7/00

(52) U.S. Cl. CPC A61K 39/12; A61K 2039/545; C12N 7/00

(57) ABSTRACT

A method of enhancing immune response in hosts, including vaccinating the hosts with a immunogenic composition, so as to enhance antibody immune response and/or cellular immune response to an immunogen. The immunogenic composition includes the immunogen and an adjuvant additive. The adjuvant additive includes a receptor associated protein (RAP) having an amino acid sequence of SEQ ID NO:1 and/or a pseudomonas exotoxin A (PE) protein.

### CROSS-REFERENCE TO RELATED APPLICATION

This application is a divisional application of and claims the priority benefit of U.S. application Ser. No. 16/801,175, filed on Feb. 26, 2020, now pending. The prior U.S. application Ser. No. 16/801,175 also claims the priority benefit of U.S. provisional application Ser. No. 62/825,834, filed on Mar. 29, 2019. The entirety of each of the above-mentioned patent applications is hereby incorporated by reference herein and made a part of this specification.

### BACKGROUND

### Technical Field

The present invention generally relates to an immunogenic composition, in particular, relates to a method of enhancing immune response in hosts by using an immunogenic composition including the adjuvant additive.

### Description of Related Art

Generally, immunogenic compositions include immunogens, adjuvants, and excipients. The immunogen includes substances that evoke an immune response. The immunogen may contain peptides, proteins or even polysaccharides. However, other ingredients in the immunogenic compositions may have influences upon the immunogenic effect of the immunogenic compositions.

### SUMMARY

Accordingly, the present disclosure is directed to an immunogenic composition including an adjuvant additive that may be used for enhancing antibody immune response and/or cellular immune response in hosts, and may provide sufficient immune protection against the immunogen.

In accordance with some embodiments of the present disclosure, an immunogenic composition is provided. The immunogenic composition includes an immunogen and an adjuvant additive. The adjuvant additive includes a receptor associated protein (RAP) having an amino acid sequence of SEQ ID NO:1 and/or a pseudomonas exotoxin A (PE) protein.

In the above embodiment, the PE protein including a target peptide has an amino acid sequence of SEQ ID NO:2.

In the above embodiment, the PE protein has an amino acid sequence of SEQ ID NO:3.

In the above embodiment, the PE protein has an amino acid sequence of SEQ ID NO:4.

In the above embodiment, the immunogen includes porcine circovirus type 2 (PCV2) virus-like particle (VLP) having an amino acid sequence of SEQ ID NO:5.

In the above embodiment, a ratio of the PCV2 VLP to the adjuvant additive is 1:0.5 by weight.

In the above embodiment, a ratio of the PCV2 VLP to the adjuvant additive is 1:1 by weight.

In the above embodiment, a ratio of the PCV2 VLP to the adjuvant additive is 1:2 by weight.

In the above embodiment, a concentration of the PCV2 VLP is about 3 μg/dose.

In the above embodiment, a concentration of the adjuvant additive is about 3 μg/dose.

In another embodiment of the present disclosure, a method of enhancing immune response in hosts is described. The method includes vaccinating the hosts with the immunogenic composition described above so as to enhance antibody immune response and/or cellular immune response to the immunogen.

In the above embodiment, the host is vaccinated by administering a dose of the immunogenic composition at least three weeks ago.

Based on the above, the present invention provides an immunogenic composition including an adjuvant additive. The adjuvant additive includes a receptor associated protein (RAP) having an amino acid sequence of SEQ ID NO:1 and/or a pseudomonas exotoxin A (PE) protein. By adding the adjuvant additive of the present disclosure in the vaccine composition for vaccination, systemic antibody immune response and/or cellular immune response may be successfully induced and enhanced, hence providing sufficient immune protection against the immunogen.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is illustrating the results from ELISA for detecting the presence of PCV2 antibody in mice for different test groups from Example 1.

FIG. 2A and FIG. 2B illustrate the results from flow cytometry for detecting the number of CD³⁺/INF-γ T cells in mice for different test groups from Example 1.

FIG. 3 illustrates the results from flow cytometry for detecting the number of CD³⁺/INF-γ T cells in mice for different test groups from Example 2.

### DESCRIPTION OF THE EMBODIMENTS

Currently, one fusion protein comprising an immunogen, a receptor-associated protein (RAP) and a Pseudomonas exotoxin A (PE) can induce a pathogen antigen-specific T cell immune response by binding to an antigen presenting cells or CD91. However, the preparation of fusion protein containing RAP and PE not only increase the difficulty of vaccine development, but also limit the applicability of RAP and PE.

The present disclosure is directed to an immunogenic composition including an adjuvant additive for enhancing immune response in hosts. In some exemplary embodiments, the adjuvant additive may at least include a receptor associated protein (RAP) having an amino acid sequence of SEQ ID NO:1 and/or a pseudomonas exotoxin A (PE) protein.

The receptor associated protein (RAP) is an antagonist and molecular chaperone that binds tightly to low-density lipoprotein receptor family members, for example, low density lipoprotein receptor-related protein 1 (LRP1), also known as cluster of differentiation 91 (CD91).

The pseudomonas exotoxin A (PE) protein is the most toxic virulence factor of this bacterium. The PE protein can be divided into Ia domain (amino acid sequence 1-252), II domain (amino acid 253-364), Ib domain (amino acid sequence 365-404) and III domain (amino acid sequence 405-613). In some embodiments, the amino acid sequence 1-407 of the PE protein (PE407) is used as part of the adjuvant additive and may have an amino acid sequence of SEQ ID NO:3. However, the disclosure is not limited thereto. In certain embodiments, a target peptide fused at the c-terminal of the PE407 is used as part of the adjuvant additive and may have an amino acid sequence of SEQ ID NO:2. For example, the target peptide including KDELKDELKDEL (referred to as K3) may fuse at the c-terminal of the PE407 (PE407-K3). Other types of target peptide may be selected and used based on actual requirement. In some embodiments, amino acid sequence 1-252 of the PE protein (PE252) is used as part of the adjuvant additive and may have an amino acid sequence of SEQ ID NO:4.

In some exemplary embodiments, the immunogenic composition may at least include an immunogen and the adjuvant additive described above. For example, the immunogen may include a porcine circovirus type 2 (PCV2) virus-like particle (VLP) having an amino acid sequence of SEQ ID NO:5. However, the disclosure is not limited thereto. Porcine circoviruses (PCVs), the smallest known animal viruses, includes PCV1 and PCV2. PCV2 was isolated from pigs suffering from post-weaning multi-systemic wasting syndrome (PMWS). PCV2-associated disease became one of the most important and economically significant swine diseases.

In one exemplary embodiment, a ratio of the PCV2 VLP to the adjuvant additive is 1:0.5 by weight. In some embodiments, a ratio of the PCV2 VLP to the adjuvant additive is 1:1 by weight. In certain embodiments, a ratio of the PCV2 VLP to the adjuvant additive is 1:2 by weight. By adjusting the ratio of the PCV2 VLP to the adjuvant additive in such a range, sufficient antibody immune response and/or cellular immune response against PCV2 VLP may be ensured.

In one exemplary embodiment, in the immunogenic composition, a concentration of the PCV2 VLP is about 3 μg/dose. In some embodiments, in the immunogenic composition, a concentration of the adjuvant additive is about 3 μg/dose.

Furthermore, in the immunogenic composition, the porcine circovirus type 2 (PCV2) virus-like particle (VLP) may be assembled from viral structural proteins and are devoid of any genetic material. In other words, PCV2 VLP is non-infectious and may be a safe alternative to inactivated infectious viruses. In one exemplary embodiment, the PCV2 VLP has an amino acid sequence of SEQ ID NO:5. However, the disclosure is not limited thereto. For example, other strains of PCV2 VLP having other amino acid sequence(s) may be used.

By designing the adjuvant additive to include at least a receptor associated protein (RAP) having an amino acid sequence of SEQ ID NO:1, and/or a pseudomonas exotoxin A (PE) protein, systemic antibody immune response and/or cellular immune response may be successfully induced by an immunogenic composition including the adjuvant additive described above, hence providing sufficient immune protection against PCV2 virus.

A method of enhancing immune response in hosts can be achieved by using the immunogenic composition described above. For example, in some embodiments, a host is vaccinated with the immunogenic composition at least three weeks ago so as to enhance antibody immune response and/or cellular immune response to the PCV2 VLP. In one exemplary embodiment, the host is vaccinated by administering a dose of the immunogenic composition at least three weeks ago. However, the disclosure is not limited thereto.

By using the method of enhancing antibody immune response and/or cellular immune response in hosts described above, sufficient immune protection against PCV2 virus may be ensured.

### EXAMPLES

The following experimental examples are performed to prove that the immunogenic composition including the adjuvant additive of the present disclosure can successfully induce antibody immune response and/or cellular immune response, so that sufficient immune protection against PCV2 virus can be conferred.

### Example 1: Effect of Adjuvant Additive PE and/or RAP on Enhancing Immune Response

In this example, the preparation of PCV2 VLP, RAP, and PE407-K3 was performed first. Specifically, the preparation method of the PCV2 VLP having an amino acid sequence of SEQ ID NO:5 was as follows: 1-4*10⁶ SF9 cells were infected with baculovirus carrying PCV2-ORF2 sequence of 0.1-1 MOI, and incubated for 3-6 days. The cell pellet was collected by centrifugation, and the PCV2 virus-like particles were extracted and purified from the cell pellet. The PCV2 virus-like particles were inactivated by 1.6-10 mM binary ethylenimine (BEI) at 37° C. for 4-48 hours so as to obtain the PCV2 VLP. The concentration of the PCV2 VLP was quantified by BSA standard.

The preparation method of the RAP having an amino acid sequence of SEQ ID NO:1 is as follows: for example, E. coli with RAP fragment was cultured to OD600 0.3-1.2, then inducing RAP fragment expression for 2-24 hours with 0.1-10 mM IPTG. The cell pellet was collected by centrifugation and then extracted with TNE buffer. After centrifugation, the supernatant of extract was purified by HIS column to obtain the RAP. The concentration of the RAP was quantified by BSA standard.

The preparation method of the PE407-K3 having an amino acid sequence of SEQ ID NO:2 is as follows: for example, E. coli with PE407-K3 fragment was cultured to OD600 0.3-1.2, then inducing PE407-K3 expression for 2-24 hours with 0.1-10 mM IPTG. The cell pellet was collected by centrifugation and then extracted with TNE buffer. After centrifugation, the pellet of extract was dissolved into 8M urea buffer. After refolding process, the PE407-K3 is obtained. The concentration of the PE407-K3 was quantified by BSA standard.

Next, the preparation of the immunogenic composition was carried out. In this experimental example, the PCV2 VLP was uniformly mixed with PE or RAP, for example, in a ratio of 1:1. However, the disclosure is not limited thereto. In other experimental examples, PCV2 VLP and PE or RAP can also be mixed uniformly in a ratio of 1:0.5 or 1:2. In this experimental example, the concentration of the PCV2 VLP is about 3 μg/dose and the concentration of the adjuvant additive is about 3 μg/dose. The ISA206 adjuvant and the uniformly mixture were warmed in a 31° C. water bath for 20 min. Then, the mixture was added to the ISA206 adjuvant at 31° C. and stirred at 400 to 700 rpm (depending on the size of the container) for 1.5 hours. After standing at room temperature for several hours, the vaccine composition was obtained and then stored at 4° C.

In this experimental example, Balb/c mice were used as test animals and were injected with immunogenic composition by subcutaneous injection. Balb/c mice were divided into 4 groups and there are 4 mice in each group as shown in Table 1. Group A was Placebo, PCV2 VLP, PE, RAP and ISA206 adjuvant were not injected. The immunogenic composition injected in group B includes PCV2 VLP and ISA206 adjuvant. The immunogenic composition injected in group C includes PCV2 VLP, RAP and ISA206 adjuvant. The immunogenic composition injected in group D includes PCV2 VLP, PE407-K3 and ISA206 adjuvant.

After injection, blood samples were collected by submandibular blood collection every week. Serum from the blood samples was used for PCV2 IgG antibody ELISA analysis. Mice were sacrificed by CO₂ three weeks after injection. Next, the spleens of the mice were taken and subjected to cellular immunostaining analysis using a flow cytometer.

TABLE 1

| Group | Immunogenic composition |
|---|---|
| A | Placebo |
| B | PCV2 VLP + ISA206 |
| C | PCV2 VLP + RAP + ISA206 |
| D | PCV2 VLP + PE407-K3 + ISA206 |

[PCV2 IgG Antibody ELISA Analysis]

Experimental procedure for the PCV2 IgG antibody ELISA analysis: the serum of 4 mice (10 μl/each mouse) of group were mixed into the same tube. After dilute each group of serum 50-fold, 100 μl of serum dilution was added to one well of the antigen plate of the Biocheck PCV2 ELISA kit, and reacted at 37° C. for 30 minutes. After washing 4 times with 1×PBST, 100 μl anti-mouse-IgG-HRP (1:10000) was added and reacted at 37° C. for 30 minutes. After washing 4 times with 1×PBST, 100 μl 3,3′,5,5′-Tetramethylbenzidine (TMB) was added and reacted at room temperature for 15 minutes. After adding 100 μl of IN H₂SO₄, the signal at a wavelength of 450 nm was measured by an ELISA reader.

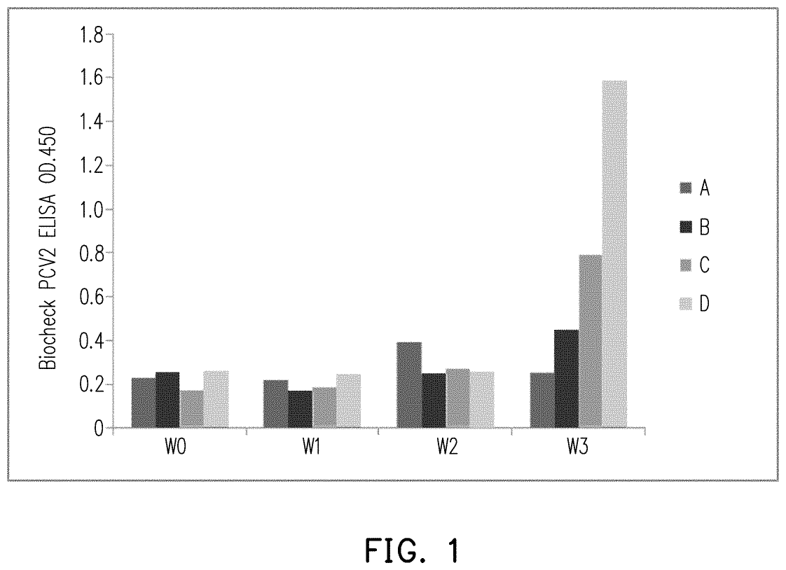

FIG. 1 illustrates the results from ELISA for detecting the presence of PCV2 antibody in mice for different test groups from Example 1. The horizontal axis represents serum of each group of mice before injection (W0), 1 week after injection (W1), 2 weeks after injection (W2), and 3 weeks after injection (W3). The vertical axis represents the optical density reading at a wavelength of 450 nm, which can represent the relative amount of PCV2 antibodies in the serum. According to the results of FIG. 1, there was no significant change in the amount of PCV2 antibody at 1 week after injection and 2 weeks after injection compared to the amount of PCV2 antibody before injection. However, at 3 weeks after injection, PCV2 antibodies from Groups C and D (injected with PCV2 VLP immunogen and adjuvant additives RAP/PE) were significantly higher than Group B (injected with PCV2 VLP immunogen only).

It can be seen that the immunogenic composition including the adjuvant additive (RAP or PE) can successfully enhance the immune response, such as the antibody immune response, to obtain sufficient immune protection against the PCV2 virus.

[Cellular Immunostaining Analysis]

Experimental procedure for cellular immunostaining analysis: Step 1. Isolation and culture of spleen cells: Three weeks after injection, the mice were sacrificed with CO₂ and blood was collected from the heart (>0.5 ml). The spleens of the mice were taken out and placed in a 24 well plate containing 1 ml/well DMEM medium. After washing once with PBS, the spleens were placed in a 6-cm dish containing 1.5 ml of RBC lysis buffer and then ground using a syringe head for 5 min. The ground spleen was passed through a cell strainer (40 um) to a 50-ml centrifuge tube, and the cell strainer was rinsed with 8.5 ml of PBS to a total volume of 10 ml. After centrifugation at 1300 rpm for 5 min, the cell pellet was collected and washed with 5 ml PBS. After centrifugation at 1300 rpm for 5 min, the cell pellet was suspended in 2 ml RPMI medium (10% FBS, 1% PSA). After cell counting, the 2*10⁷ spleen cells/well were seeded in 6-well plate and cultured in 2 ml RPMI medium (10% FBS, 1% PSA). The 2 μg PCV2-ORF2 immunogen (PCV2 VLP immunogen) was added to react for 16 hours. The PCV2 VLP immunogen can stimulate CD³⁺ T cells of spleen cells to produce IFN-γ. Step 2. Immunostaining of spleen cells: After adding the Golgi plug and reacting for 4 hours, the cells were taken to a centrifuge tube. After centrifugation at 300×g, 4° C., 5 min, the supernatant was removed and 1 ml PBS was added for washing. After washing with 1 ml PBS again, 100 μl/well of the antibodies specific for CD3, CD4, and CD8 were added and incubated at 4° C. for 30 mins in the dark. After washing twice with 1 ml PBS, the 200 μl IC Fixation solution (PBS: IC Fix=1:1) was added and reacted at 4° C. for overnight in the dark. After washing with 1 ml permeabilization wash buffer, 100 μl IFN-γ-Ab was added to react for 30 min at room temperature in the dark. Finally, detecting the number of CD³⁺ IFN-γ T cells by flow cytometry.

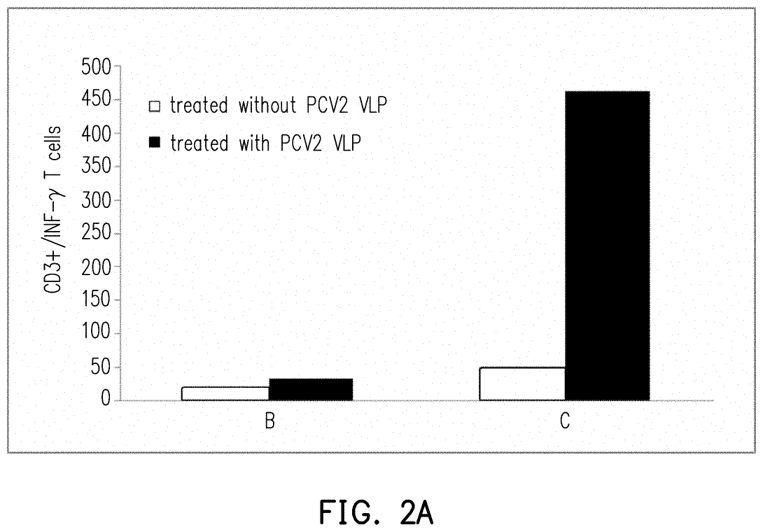

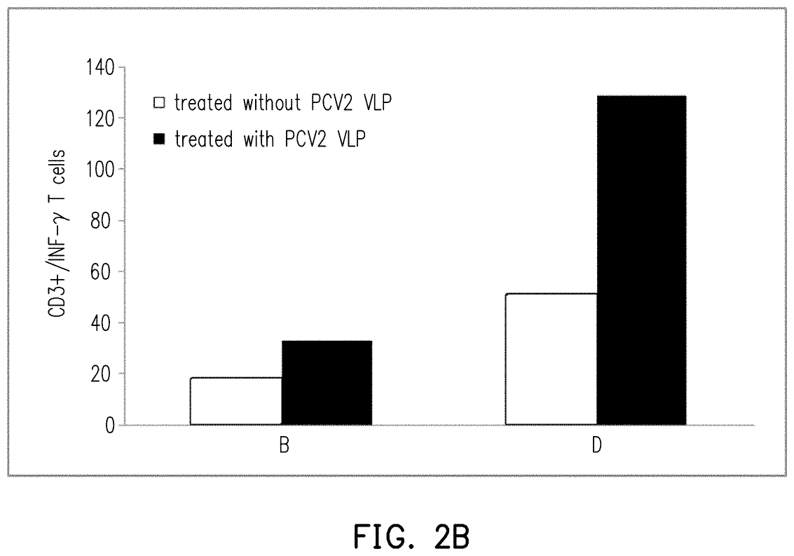

FIG. 2A and FIG. 2B illustrate the results from flow cytometry for detecting the number of CD³⁺/INF-γ T cells in mice for different test groups from Example 1. The horizontal axis represents the spleen cells of each group of mice at 3 weeks after injection treated with PCV2 VLP immunogen (black column) or without PCV2 VLP immunogen (white column). The vertical axis represents the number of CD³⁺/INF-γ T cells. According to the results of FIG. 2A, compared with the spleen cells of the B group injected with PCV2 VLP only, the spleen cells of group C injected with PCV2 VLP+RAP can produce more specific CD³⁺/INF-γ T cells after stimulated by PCV2 VLP immunogen.

According to the results of FIG. 2B, compared with the spleen cells of the B group injected with PCV2 VLP only, the spleen cells of group D injected with PCV2 VLP+PE407-K3 can produce more specific CD³⁺/INF-γ T cells after stimulated by PCV2 VLP immunogen.

It can be seen that the immunogenic composition including the adjuvant additive (RAP or PE) can successfully enhance the immune response, such as the cellular immune response, to obtain sufficient immune protection against the PCV2 virus.

### Example 2: Effect of Different PE Fragments on Increasing Cellular Immune Response

In this example, the preparation of PCV2 VLP and different PE fragments (PE252, PE407 and PE407-K3) was performed first. Specifically, the preparation method of the PCV2 VLP having the amino acid sequence of SEQ ID NO: 5 is already described in Example 1. The preparation method of the PE407-K3 having the amino acid sequence of SEQ ID NO: 2 is also already described in Example 1. Further, since the preparation method of PE407 having the amino acid sequence of SEQ ID NO: 3 and PE252 having the amino acid sequence of SEQ ID NO: 4 is similar to the preparation method of PE407-K3, it will not be described herein.

In addition, since the preparation method of the immunogenic composition is similar to that of Example 1, it will not be described herein. In this experimental example, the PCV2 VLP was uniformly mixed with PE or RAP, for example, in a ratio of 1:1. The concentration of the PCV2 VLP is about 3 μg/dose and the concentration of the adjuvant additive (PE252, PE407 and PE407-K3) is about 3 μg/dose.

In this experimental example, Balb/c mice were used as test animals and were injected with immunogenic composition by subcutaneous injection. Balb/c mice were divided into 4 groups and there are 4 mice in each group as shown in Table 2. The immunogenic composition injected in group A includes PCV2 VLP and ISA206 adjuvant. The immunogenic composition injected in group B includes PCV2 VLP, PE252 and ISA206 adjuvant. The immunogenic composition injected in group C includes PCV2 VLP, PE407 and ISA206 adjuvant. The immunogenic composition injected in group D includes PCV2 VLP, PE407-K3 and ISA206 adjuvant.

TABLE 2

| Group | Immunogenic composition |
|---|---|
| A | PCV2 VLP + ISA206 |
| B | PCV2 VLP + PE252 + ISA206 |
| C | PCV2 VLP + PE407 + ISA206 |
| D | PCV2 VLP + PE407-K3 + ISA206 |

Mice were sacrificed by CO₂ three weeks after injection. The spleens of the mice were taken and subjected to cellular immunostaining analysis using a flow cytometer. In addition, since the experimental procedure of the cellular immunostaining analysis is similar to that of Example 1, it will not be described herein.

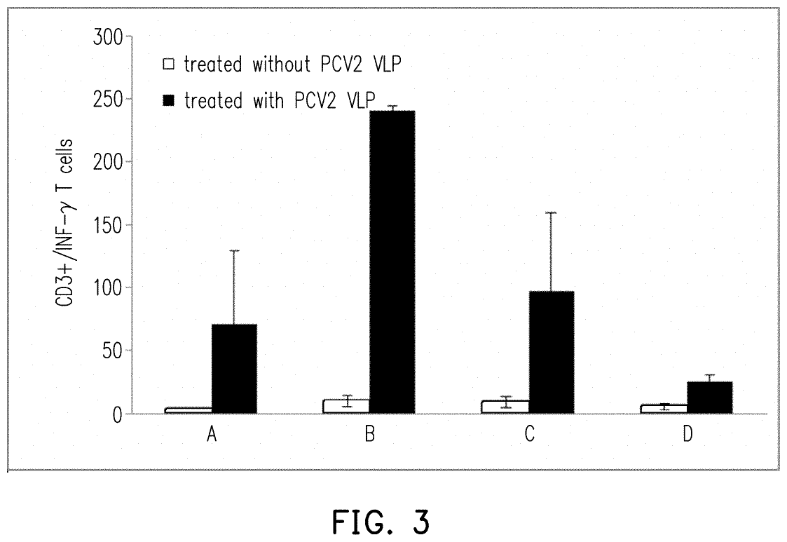

FIG. 3 illustrates the results from flow cytometry for detecting the number of CD³⁺/INF-γ T cells in mice for different test groups from Example 2. The horizontal axis represents the spleen cells of each group of mice at 3 weeks after injection treated with PCV2 VLP immunogen (black column) or without PCV2 VLP immunogen (white column). The vertical axis represents the number of CD³⁺/INF-γ T cells. According to the results of FIG. 3, compared with the spleen cells of the A group injected with PCV2 VLP only, the spleen cells of group B injected with PCV2 VLP+PE252 can produce more specific CD³⁺//INF-γ T cells after stimulated by PCV2 VLP immunogen. In addition, the number of specific CD3+/IFN-γ T cells stimulated by PCV2 VLP immunogen in group B was significantly higher than that of group C (injected with PCV2 VLP+PE407) and group D (injected with PCV2 VLP+PE407-K3).

It can be seen that the immunogenic composition including the adjuvant additive (PE252, PE407 and PE407-K3) can successfully enhance the immune response, such as the cellular immune response, to obtain sufficient immune protection against the PCV2 virus.

It is worth noting that although Examples 1 and 2 are adding the adjuvant additive (RAP and/or PE) to the PCV2 VLP immunogen to enhance the antibody immune response and the cellular immune response against the PCV2 VLP, the disclosure is not limited thereto. In other embodiments, the adjuvant additive (RAP and/or PE) may also be added to other immunogens to enhance the host's antibody immune response and cellular immune response against the infectious pathogen.

According to the above embodiments, the adjuvant additive of the present disclosure including the receptor associated protein (RAP) having an amino acid sequence of SEQ ID NO:1 or a pseudomonas exotoxin A (PE) protein is used to add into the immunogenic composition to induce and enhance antibody immune response and/or cellular immune response, hence providing sufficient immune protection against the immunogen. In addition, a synergistic effect of further enhancing the immune response may be observed when at least the RAP and PE are used together in a single immunogenic composition.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A method of enhancing immune response in hosts, comprising:
vaccinating the hosts with a immunogenic composition, so as to enhance antibody immune response and/or cellular immune response to an immunogen,
wherein the immunogenic composition comprises the immunogen and an adjuvant additive, and the adjuvant additive includes at least one selected from a group consisting of a receptor associated protein (RAP) having an amino acid sequence of SEQ ID NO:1 and a pseudomonas exotoxin A (PE) protein.

2. The method of enhancing immune response in the hosts according to claim 1, wherein the host is vaccinated by administering a dose of the immunogenic composition at least three weeks ago.


SEQUENCE LISTING (5 sequences)

SEQ ID NO: 1 - protein, 105 aa, Homo sapiens
MAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRAR

SEQ ID NO: 2 - protein, 424 aa, Pseudomonas aeruginosa
MAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVEFLEKDELKDELKDEL

SEQ ID NO: 3 - protein, 407 aa, Pseudomonas aeruginosa
MAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGD

SEQ ID NO: 4 - protein, 252 aa, Pseudomonas aeruginosa
MAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFP

SEQ ID NO: 5 - protein, 233 aa, Porcine circovirus
MTYPRRRYRRRRHRPRSHLGQILRRRPWLVHPRHRYRWRRKNGIFNSRLSRTFGYTVKATTVTTPSWAVDMLRFNIDDFLPPGGGTNKISIPFEYYRIRKVKVEFWPCSPITQGDRGVGSSAVILDDNFFPKSTALTYDPYVNYSSRHTIPQPFSYHSRYFTPKPVLDSTIDYFQPNNKRNQLWMRIQTSKNVDHVGLGTAFENSKYDQDYNIRVTMYVQFREFNLKDPPLKP